(19) United States
(12) Patent Application Publication
(10) Pub. No.: US 2024/0207345 A1
(43) Pub. Date: Jun. 27, 2024

(54) METHODS AND COMPOSITIONS FOR TREATING HAIR LOSS

(71) Applicant: Erkkie Harris-Wells, Costa Mesa, CA (US)

(72) Inventor: Erkkie Harris-Wells, Costa Mesa, CA (US)

(21) Appl. No.: 18/425,259

(22) Filed: Jan. 29, 2024

Related U.S. Application Data
(63) Continuation-in-part of application No. 18/088,253, filed on Dec. 23, 2022, now abandoned.

(51) Int. Cl. A61K 36/48; A61K 9/06; A61K 31/047; A61K 31/728; A61K 35/74; A61K 36/064; A61K 36/185; A61K 36/22; A61K 36/53; A61K 36/61; A61K 47/10; A61K 47/18; A61K 47/38

(52) U.S. Cl. CPC A61K 36/48; A61K 9/06; A61K 31/047; A61K 31/728; A61K 35/74; A61K 36/064; A61K 36/185; A61K 36/22; A61K 36/53; A61K 36/61; A61K 47/10; A61K 47/183; A61K 47/38

(57) ABSTRACT

The disclosed subject matter pertains to methods for treating hyperhidrosis in a patient. The methods involve preparing a shampoo or lotion with specific formulations and applying it to the patient's scalp. The methods also involve diagnosing hyperhidrosis by extracting hair bulbs from the patient, cleaning and treating the hair bulbs for microscopic analysis, viewing an accumulation of metabolic waste, and neutralizing the metabolic waste.

### CROSS-REFERENCE TO RELATED APPLICATIONS

“This application is a Continuation-in-Part Utility Patent application claiming priority to U.S. Utility patent application Ser. No. 18/088,253, filed on Dec. 23, 2022, which is incorporated by reference herein in its entirety.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1. Field of the Invention

The present invention generally relates to methods and compositions for diagnosing and treating hyperhidrosis, and more specifically, methods including the preparation and application of specialized shampoos and lotions.

### 2. Description of Related Art

The prior art teaches a range of compositions that are useful for treating hair loss. For example, Esco, U.S. Pat. No. 10,226,495, teaches a composition for promoting hair growth that includes hinoki oil, red clover extract, and a peptide. These prior art products are typically targeted to DHT, and not hyperhidrosis (excessive sweating), which can be a significant contributing factor towards alopecia.

Hyperhidrosis is a medical condition characterized by excessive sweating, which can occur in various parts of the body such as the hands, feet, armpits, and the scalp. This condition can cause discomfort, social embarrassment, and can interfere with daily activities. Various treatments have been proposed to manage this condition, including topical applications, oral medications, and surgical interventions.

Topical applications for treating hyperhidrosis typically include antiperspirants containing aluminum salts, creams, and lotions. These products work by blocking the sweat ducts, thereby reducing the amount of sweat that reaches the skin's surface. However, these products may not be suitable for all areas of the body, such as the scalp, and may cause skin irritation in some individuals.

Shampoos and lotions are common products used for scalp care. They are typically formulated with various ingredients to cleanse the scalp, remove excess oil, and provide hydration. These products may also contain active ingredients to address specific scalp conditions such as dandruff, seborrheic dermatitis, and psoriasis. The formulation of these products can greatly influence their effectiveness and user experience.

Ingredients commonly used in shampoos and lotions include surfactants, moisturizers, preservatives, and various plant extracts. Surfactants are used to remove dirt and oil from the scalp, while moisturizers help to hydrate the skin. Preservatives are added to prevent microbial growth in the product. Plant extracts are often included for their potential beneficial effects on the skin, such as anti-inflammatory, antioxidant, and skin-soothing properties.

Adaptogens are a class of natural substances that are believed to help the body adapt to stress and exert a normalizing effect upon bodily processes. They have been used in traditional medicine for centuries and are now being incorporated into modern skincare products for their potential skin health benefits. Some examples of adaptogens include propanediol, glycerin, and Phyllanthus Emblica Fruit extract.

Despite the variety of available treatments and products, managing hyperhidrosis remains a challenge for many individuals. Therefore, there is a continuous search for new methods and compositions that can effectively treat this condition.

### SUMMARY OF INVENTION

In general, in a first aspect, the subject matter features a method for treating hyperhidrosis in a patient. This method involves preparing a shampoo in a mixing vessel. The shampoo preparation includes adding and mixing a first formulation having 40-90% water by weight, 10-50% surfactant by weight, 1-5% amino acid derived cleansing and skin softening agent by weight, and 1-5% glycerin-free amphoteric surfactant by weight. A second formulation is also added and mixed, which includes 0.05-3.00% Vitamin B moisturizer by weight, 0.05-3.00% saccharomyces ferment by weight, 0.01-1.00% Caesalpinia spinosa extract by weight, 0.01-1.00% of an adaptogen including propanediol, glycerin and Phyllanthus Emblica Fruit extract, 0.05-5.00% of a skin hydration composition by weight, 0.05-5.00% Pisum sativum sprout extract by weight, 0.05-5.00% Ocimum basilicum hairy root culture extract by weight, 0.05-5.00% pseudoalteromonas ferment extract by weight, and 0.1-5.00% of a preservative by weight. The shampoo is then applied to a scalp of the patient and rinsed from the head of the patient. The weight percentages are based on the total weight of the shampoo.

Embodiments of the subject matter may include one or more of the following features. The shampoo may include Schinus terebinthifolius Raddi or Anetholea 45 leaf extract.

In another aspect, the subject matter features a method for treating hyperhidrosis in a patient by preparing a lotion in a mixing vessel. The lotion preparation includes adding and mixing a first formulation having 40-95% water by weight and 0.05-3.00% hydroxyethylcellulose. Other ingredients are also added and mixed, including 0.01-2.00% tetrasodium glutamate diacetate by weight, 0.01-1.00% lactic acid by weight, 0.1-5% of a fourth formulation including glycerin, sodium hyaluronate, phenoxyethanol, and ethylhexlglycerin, 0.001-1.00% hydrolyzed sodium hyaluronate by weight, 0.05-5.00% Epilobium angustifolium flower extract by weight, 0.02-5.00% saccharomyces ferment by weight, 0.01-1.00% of an adaptogen including propanediol, glycerin and Phyllanthus Emblica Fruit extract, 0.02-3.00% Caesalpinia spinosa extract by weight, 0.5-5.00% Pisum sativum sprout extract by weight, 0.05-5.00% Ocimum basilicum hairy root culture extract by weight, 0.05-5.00% pseudoalteromonas ferment extract by weight, 0.1-5.00% phenoxyethanol by weight, and 0.1-5.00% ethylhexlglycerin by weight. The lotion is then applied to a scalp of the patient. The weight percentages are based on the total weight of the lotion.

Embodiments of the subject matter may include one or more of the following features. The lotion may include Schinus terebinthifolius Raddi or Anetholea anista leaf extract.

In yet another aspect, the subject matter features a method for diagnosing and treating hyperhidrosis in a patient. This method involves extracting 15 to 20 hair bulbs from the patient, cleaning and treating the hair bulbs for microscopic analysis, using a polarized eye piece for viewing an accumulation of metabolic waste, and neutralizing the metabolic waste with the steps of preparing a shampoo or a lotion as described above and applying it to a scalp of the patient.

Embodiments of the subject matter may include one or more of the following features. The shampoo or lotion used for neutralizing the metabolic waste may include Schinus terebinthifolius Raddi or Anetholea anista leaf extract.

Hyperhidrosis is a medical condition characterized by excessive sweating, which can occur in various parts of the body such as the hands, feet, armpits, and the scalp. This condition can cause discomfort, social embarrassment, and can interfere with daily activities. Various treatments have been proposed to manage this condition, including topical applications, oral medications, and surgical interventions.

Topical applications for treating hyperhidrosis typically include antiperspirants containing aluminum salts, creams, and lotions. These products work by blocking the sweat ducts, thereby reducing the amount of sweat that reaches the skin's surface. However, these products may not be suitable for all areas of the body, such as the scalp, and may cause skin irritation in some individuals.

Shampoos and lotions are common products used for scalp care. They are typically formulated with various ingredients to cleanse the scalp, remove excess oil, and provide hydration. These products may also contain active ingredients to address specific scalp conditions such as dandruff, seborrheic dermatitis, and psoriasis. The formulation of these products can greatly influence their effectiveness and user experience.

Ingredients commonly used in shampoos and lotions include surfactants, moisturizers, preservatives, and various plant extracts. Surfactants are used to remove dirt and oil from the scalp, while moisturizers help to hydrate the skin. Preservatives are added to prevent microbial growth in the product. Plant extracts are often included for their potential beneficial effects on the skin, such as anti-inflammatory, antioxidant, and skin-soothing properties.

Adaptogens are a class of natural substances that are believed to help the body adapt to stress and exert a normalizing effect upon bodily processes. They have been used in traditional medicine for centuries and are now being incorporated into modern skincare products for their potential skin health benefits. Some examples of adaptogens include propanediol, glycerin, and Phyllanthus Emblica Fruit extract.

Despite the variety of available treatments and products, managing hyperhidrosis remains a challenge for many individuals. Therefore, there is a continuous search for new methods and compositions that can effectively treat this condition.

Embodiments of the subject matter may include one or more of the following features. The shampoo may include Schinus terebinthifolius Raddi or Anetholea anista leaf extract.

In another aspect, the subject matter features a method for treating hyperhidrosis in a patient by preparing a lotion in a mixing vessel. The lotion preparation includes adding and mixing a first formulation having 40-95% water by weight and 0.05-3.00% hydroxyethylcellulose. Other ingredients are also added and mixed, including 0.01-2.00% tetrasodium glutamate diacetate by weight, 0.01-1.00% lactic acid by weight, 0.1-5% of a fourth formulation including glycerin, sodium hyaluronate, phenoxyethanol, and ethylhexlglycerin, 0.001-1.00% hydrolyzed sodium hyaluronate by weight, 0.05-5.00% Epilobium angustifolium flower extract by weight, 0.02-5.00% saccharomyces ferment by weight, 0.01-1.00% of an adaptogen including propanediol, glycerin and Phyllanthus embilica fruit extract, 0.02-3.00% Caesalpinia spinosa extract by weight, 0.5-5.00% Pisum sativum sprout extract by weight, 0.05-5.00% Ocimum basilicum hairy root culture extract by weight, 0.05-5.00% pseudoalteromonas ferment extract by weight, 0.1-5.00% phenoxyethanol by weight, and 0.1-5.00% ethylhexlglycerin by weight. The lotion is then applied to a scalp of the patient. The weight percentages are based on the total weight of the lotion.

Embodiments of the subject matter may include one or more of the following features. The lotion may include Schinus terebinthifolius Raddi or Anetholea anista leaf extract.

In yet another aspect, the subject matter features a method for diagnosing and treating hyperhidrosis in a patient. This method involves extracting 15 to 20 hair bulbs from the patient, cleaning and treating the hair bulbs for microscopic analysis, using a polarized eye piece for viewing an accumulation of metabolic waste, and neutralizing the metabolic waste with the steps of preparing a shampoo or a lotion as described above and applying it to a scalp of the patient.

Embodiments of the subject matter may include one or more of the following features. The shampoo or lotion used for neutralizing the metabolic waste may include Schinus terebinthifolius Raddi or Anetholea anista leaf extract.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The present disclosure provides methods for treating hyperhidrosis, a condition characterized by excessive sweating, using specially formulated shampoo and lotion. These methods may involve the preparation of a shampoo or a lotion with specific formulations, and their application to the scalp of a patient suffering from hyperhidrosis.

The method may involve preparing a shampoo in a mixing vessel. The shampoo may include a first formulation and a second formulation. The first formulation may include 40-90% water by weight, 10-50% surfactant by weight, 1-5% amino acid derived cleansing and skin softening agent by weight, and 1-5% glycerin-free amphoteric surfactant by weight. The second formulation may include 0.05-3.00% Vitamin B moisturizer by weight, 0.05-3.00% saccharomyces ferment by weight, 0.01-1.00% Caesalpinia spinosa extract by weight, 0.01-1.00% of an adaptogen including propanediol, glycerin and Phyllanthus Emblica Fruit extract, 0.05-5.00% of a skin hydration composition by weight, 0.05-5.00% Pisum sativum sprout extract by weight, 0.05-5.00% Ocimum basilicum hairy root culture extract by weight, 0.05-5.00% pseudoalteromonas ferment extract by weight, and 0.1-5.00% of a preservative by weight. The shampoo may be applied to the scalp of the patient and then rinsed off. The weight percentages are based on the total weight of the shampoo.

The method may involve preparing a lotion in a mixing vessel. The lotion may include a first formulation and a fourth formulation. The first formulation may include 40-95% water by weight and 0.05-3.00% hydroxyethylcellulose. The fourth formulation may include glycerin, sodium hyaluronate, phenoxyethanol, and ethylhexlglycerin. The lotion may also include 0.001-1.00% hydrolyzed sodium hyaluronate by weight, 0.05-5.00% Epilobium angustifolium flower extract by weight, 0.02-5.00% saccharomyces ferment by weight, 0.01-1.00% of an adaptogen including propanediol, glycerin and Phyllanthus Emblica Fruit extract, 0.02-3.00% Caesalpinia spinosa extract by weight, 0.5-5.00% Pisum sativum sprout extract by weight, 0.05-5.00% Ocimum basilicum hairy root culture extract by weight, 0.05-5.00% pseudoalteromonas ferment extract by weight, 0.1-5.00% phenoxyethanol by weight, and 0.1-5.00% ethylhexlglycerin by weight. The lotion may be applied to the scalp of the patient. The weight percentages are based on the total weight of the lotion.

The shampoo or lotion may include Schinus terebinthifolius Raddi or Anetholea anista leaf extract. In other cases, the method may also involve diagnosing hyperhidrosis in a patient by extracting 15 to 20 hair bulbs from the patient, cleaning and treating the hair bulbs for microscopic analysis, using a polarized eye piece for viewing an accumulation of metabolic waste, and neutralizing the metabolic waste with the specially formulated shampoo or lotion.

The method may involve preparing a shampoo in a mixing vessel. The shampoo may include a first formulation and a second formulation. The first formulation may include 40-90% water by weight, 10-50% surfactant by weight, 1-5% amino acid derived cleansing and skin softening agent by weight, and 1-5% glycerin-free amphoteric surfactant by weight. The second formulation may include 0.05-3.00% Vitamin B moisturizer by weight, 0.05-3.00% saccharomyces ferment by weight, 0.01-1.00% Caesalpinia spinosa extract by weight, 0.01-1.00% of an adaptogen including propanediol, glycerin and Phyllanthus Emblica Fruit extract, 0.05-5.00% of a skin hydration composition by weight, 0.05-5.00% Pisum sativum sprout extract by weight, 0.05-5.00% Ocimum basilicum hairy root culture extract by weight, 0.05-5.00% pseudoalteromonas ferment extract by weight, and 0.1-5.00% of a preservative by weight.

The shampoo may include Schinus terebinthifolius Raddi. Schinus terebinthifolius Raddi, also known as Brazilian pepper tree, may be included in the shampoo formulation for its potential benefits to the skin and hair. In other cases, the shampoo may include Anetholea anista leaf extract. Anetholea anista, also known as aniseed myrtle, may be included in the shampoo formulation for its potential benefits to the skin and hair. The inclusion of these ingredients may provide additional benefits to the patient, such as improved scalp health and hair quality.

The shampoo may be prepared by adding and mixing the ingredients of the first formulation in a mixing vessel, followed by the addition and mixing of the ingredients of the second formulation. The mixing may be performed until a homogeneous mixture is obtained. The shampoo may then be applied to the scalp of the patient and then rinsed off. The weight percentages are based on the total weight of the shampoo.

The method may involve applying the prepared shampoo to the scalp of the patient. The application process may involve massaging the shampoo into the scalp, ensuring that all areas of the scalp are covered. This may help in the effective distribution of the shampoo and its active ingredients across the scalp. The shampoo may be left on the scalp for a period of time to allow for the absorption of the active ingredients. The duration of this period may vary depending on the severity of the hyperhidrosis condition and the specific formulation of the shampoo.

Following the application of the shampoo, the method may involve rinsing the shampoo from the scalp of the patient. The rinsing process may involve the use of warm water to thoroughly remove the shampoo from the scalp and hair. The rinsing process may be repeated as many times as is deemed appropriate to ensure that all the shampoo is removed from the scalp. This may help in preventing any potential irritation or discomfort that may be caused by the residual shampoo on the scalp.

In some cases, the application and rinsing process may be repeated multiple times during a single treatment session. The frequency and duration of the treatment sessions may be adjusted based on the patient's response to the treatment and the severity of the hyperhidrosis condition. In other cases, the shampoo may be used in conjunction with other treatments or therapies for hyperhidrosis, providing a comprehensive approach to managing and treating the condition.

In some variations, the shampoo may include Schinus terebinthifolius Raddi. Schinus Terebinthifolius Raddi, also known as Brazilian pepper tree, may be included in the shampoo formulation for its potential benefits to the skin and hair. The inclusion of Schinus Terebinthifolius Raddi may provide additional benefits to the patient, such as improved scalp health and hair quality.

In other variations, the shampoo may include Anetholea anista leaf extract. Anetholea anista, also known as aniseed myrtle, may be included in the shampoo formulation for its potential benefits to the skin and hair. The inclusion of Anetholea anista leaf extract may provide additional benefits to the patient, such as improved scalp health and hair quality.

The inclusion of these ingredients in the shampoo formulation may not be limited to Schinus terebinthifolius Raddi and Anetholea anista leaf extract. Other ingredients known to those skilled in the art for their beneficial properties to the skin and hair may also be included in the shampoo formulation. The specific selection and combination of ingredients may be adjusted based on the specific requirements of the patient and the desired therapeutic effect.

In some aspects, the method may involve preparing a lotion in a mixing vessel. The lotion may include a first formulation and a fourth formulation. The first formulation may include 40-95% water by weight and 0.05-3.00% hydroxyethylcellulose. The fourth formulation may include glycerin, sodium hyaluronate, phenoxyethanol, and ethylhexlglycerin. The lotion may also include 0.001-1.00% hydrolyzed sodium hyaluronate by weight, 0.05-5.00% Epilobium angustifolium flower extract by weight, 0.02-5.00% saccharomyces ferment by weight, 0.01-1.00% of an adaptogen including propanediol, glycerin and Phyllanthus Emblica Fruit extract, 0.02-3.00% Caesalpinia spinosa extract by weight, 0.5-5.00% Pisum sativum sprout extract by weight, 0.05-5.00% Ocimum basilicum hairy root culture extract by weight, 0.05-5.00% pseudoalteromonas ferment extract by weight, 0.1-5.00% phenoxyethanol by weight, and 0.1-5.00% ethylhexlglycerin by weight.

The lotion may be prepared by adding and mixing the ingredients of the first formulation in a mixing vessel, followed by the addition and mixing of the ingredients of the fourth formulation. The mixing may be performed until a homogeneous mixture is obtained. The weight percentages are based on the total weight of the lotion.

The lotion may include Schinus terebinthifolius Raddi. Schinus terebinthifolius Raddi, also known as Brazilian pepper tree, may be included in the lotion formulation for its potential benefits to the skin and hair. In other cases, the lotion may include Anetholea anista leaf extract. Anetholea anista, also known as aniseed myrtle, may be included in the lotion formulation for its potential benefits to the skin and hair. The inclusion of these ingredients may provide additional benefits to the patient, such as improved scalp health and hair quality.

The inclusion of these ingredients in the lotion formulation may not be limited to Schinus terebinthifolius Raddi and Anetholea anista leaf extract. Other ingredients known to those skilled in the art for their beneficial properties to the skin and hair may also be included in the lotion formulation. The specific selection and combination of ingredients may be adjusted based on the specific requirements of the patient and the desired therapeutic effect.

The method may involve applying the prepared lotion to the scalp of the patient. The application process may involve massaging the lotion into the scalp, ensuring that all areas of the scalp are covered. This may help in the effective distribution of the lotion and its active ingredients across the scalp. The lotion may be left on the scalp for a period of time to allow for the absorption of the active ingredients. The duration of this period may vary depending on the severity of the hyperhidrosis condition and the specific formulation of the lotion.

Following the application of the lotion, the lotion may be left on the scalp without the immediate rinsing off. This may allow for a prolonged contact of the active ingredients with the scalp, potentially enhancing their therapeutic effect. The duration of this period may be adjusted based on the patient's response to the treatment and the severity of the hyperhidrosis condition.

The lotion application process may be repeated multiple times during a single treatment session. The frequency and duration of the treatment sessions may be adjusted based on the patient's response to the treatment and the severity of the hyperhidrosis condition. In other cases, the lotion may be used in conjunction with other treatments or therapies for hyperhidrosis, providing a comprehensive approach to managing and treating the condition.

In some variations, the lotion may include Schinus terebinthifolius Raddi. for its potential benefits to the skin and hair. The inclusion of Schinus terebinthifolius Raddi in the lotion formulation may provide additional benefits to the patient, such as improved scalp health and hair quality.

In other variations, the lotion may include Anetholea anista leaf extract. Anetholea anista, also known as aniseed myrtle, may be included in the lotion formulation for its potential benefits to the skin and hair. The inclusion of Anetholea anista leaf extract in the lotion formulation may provide additional benefits to the patient, such as improved scalp health and hair quality.

The inclusion of these ingredients in the lotion formulation may not be limited to Schinus terebinthifolius Raddi and Anetholea anista leaf extract. Other ingredients known to those skilled in the art for their beneficial properties to the skin and hair may also be included in the lotion formulation. The specific selection and combination of ingredients may be adjusted based on the specific requirements of the patient and the desired therapeutic effect.

The method may also involve diagnosing hyperhidrosis in a patient. This diagnostic process may include extracting 15 to 20 hair bulbs from the patient. The extraction of hair bulbs may be performed using techniques known to those skilled in the art. The extracted hair bulbs may then be cleaned and treated for microscopic analysis. The cleaning and treatment process may involve the use of various solutions and techniques to ensure that the hair bulbs are properly prepared for analysis.

Following the cleaning and treatment of the hair bulbs, the method may involve using a polarized eye piece for viewing an accumulation of metabolic waste. The use of a polarized eye piece may enhance the visibility of the metabolic waste, allowing for a more accurate diagnosis of hyperhidrosis. The accumulation of metabolic waste may be indicative of the severity of the hyperhidrosis condition, providing valuable information for the subsequent treatment process.

The method may involve neutralizing the metabolic waste with the specially formulated shampoo or lotion. The neutralization process may involve the application of the shampoo or lotion to the scalp of the patient, followed by a rinsing process in the case of the shampoo. The active ingredients in the shampoo or lotion may interact with the metabolic waste, potentially reducing its accumulation and alleviating the symptoms of hyperhidrosis. The specific selection and combination of ingredients in the shampoo or lotion may be adjusted based on the specific requirements of the patient and the desired therapeutic effect.

The method may involve neutralizing the metabolic waste using the specially formulated shampoo or lotion. The neutralization process may involve the application of the shampoo or lotion to the scalp of the patient. The active ingredients in the shampoo or lotion may interact with the metabolic waste, potentially reducing its accumulation and alleviating the symptoms of hyperhidrosis.

The shampoo may be used for the neutralization process. The shampoo may be applied to the scalp of the patient, ensuring that all areas of the scalp are covered. This may help in the effective distribution of the shampoo and its active ingredients across the scalp. The shampoo may be left on the scalp for a period of time to allow for the absorption of the active ingredients. The duration of this period may vary depending on the severity of the hyperhidrosis condition and the specific formulation of the shampoo. Following the application of the shampoo, the method may involve rinsing the shampoo from the scalp of the patient. The rinsing process may involve the use of warm water to thoroughly remove the shampoo from the scalp and hair.

In other cases, the lotion may be used for the neutralization process. The lotion may be applied to the scalp of the patient, ensuring that all areas of the scalp are covered. This may help in the effective distribution of the lotion and its active ingredients across the scalp. The lotion may be left on the scalp for a period of time to allow for the absorption of the active ingredients. The duration of this period may vary depending on the severity of the hyperhidrosis condition and the specific formulation of the lotion. Unlike the shampoo, the lotion may be left on the scalp without the immediate rinsing off. This may allow for a prolonged contact of the active ingredients with the scalp, potentially enhancing their therapeutic effect.

The specific selection and combination of ingredients in the shampoo or lotion may be adjusted based on the specific requirements of the patient and the desired therapeutic effect. The active ingredients in the shampoo or lotion may interact with the metabolic waste, potentially reducing its accumulation and alleviating the symptoms of hyperhidrosis. This neutralization process may provide a comprehensive approach to managing and treating the condition of hyperhidrosis. Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

Another aspect of the present invention is directed to a method for treating hyperhidrosis in a patient. The method includes preparing a shampoo in a mixing vessel. The preparation of the shampoo includes adding a first mixture including 40-90% water by weight, 10-50% surfactant by weight, 1-5% amino acid derived cleansing and skin softening agent by weight and 1-5% glycerin-free amphoteric surfactant by weight. The preparation of the shampoo includes adding and mixing a second mixture including 0.05-3.00% Vitamin B % moisturizer by weight, 0.05-3.00% saccharomyces ferment by weight, 0.01-1.00% Caesalpinia spinosa extract by weight, 0.01-1.00% of an adaptogen including propanediol, glycerin and Phyllanthus Emblica Fruit extract, 0.05-5.00% of a skin hydration composition by weight, 0.05-5.00% Pisum sativum sprout extract by weight, 0.05-5.00% Ocimum basilicum hairy root culture extract by weight and 0.05-5.00% pseudoalteromonas ferment extract by weight. The preparation of the shampoo includes adding and mixing 0.1-5.00% of a preservative by weight. The method for treating hyperhidrosis in a patient includes applying the shampoo to a scalp of the patient and rinsing the shampoo from the scalp of the patient. All weight percentages are based on the total weight of the shampoo. The shampoo may include Schinus terebinthifolius Raddi or Anetholea anista leaf extract or both. The method may include extracting 15 to 20 hair bulbs from the patient, cleaning and treating the hair bulbs for microscopic analysis, using a polarized eye piece for viewing an accumulation of metabolic waste and neutralizing the metabolic waste. neutralizing the metabolic waste includes applying the shampoo to the scalp of the patient and rinsing the shampoo from the scalp of the patient.

Another aspect of the present invention is directed to a method for treating hyperhidrosis in a patient. The method includes preparing a lotion in a mixing vessel. The preparation includes adding a first mixture including 40-95% water by weight and 0.05-3.00% hydroxyethylcellulose. The preparation includes adding and mixing to the mixing vessel 0.01-2.00% tetrasodium glutamate diacetate by weight and adding and mixing to the mixing vessel 0.01-1.00% lactic acid by weight. The preparation includes preparing a second mixture including a 0.1-5.00% formulation of glycerin, sodium hyaluronate, phenoxyethanol and ethylhexlglycerin by weight, 0.001-1.00% hydrolyzed sodium hyaluronate by weight, 0.05-5.00% Epilobium angustifolium flower extract by weight, 0.02-5.00% saccharomyces ferment by weight, 0.01-1.00% of an adaptogen including propanediol, glycerin and Phyllanthus Emblica Fruit extract by weight, a 0.02-3.00% formulation of Caesalpinia spinosa extract, helianthuss annuus sprout extract, propylene glycol and sodium benzoate by weight, 0.5-5.00% Pisum sativum sprout extract by weight, a 0.05-5.00% formulation including Ocimum basilicum hairy root culture extract by weight and 0.05-5.00% pseudoalteromonas ferment extract by weight. The preparation includes adding and mixing the second mixture to the mixing vessel and preparing a third mixture including phenoxyethanol and ethylhexlglycerin. The preparation includes adding and mixing to the mixing vessel 0.1-5.00% of the third mixture by weight. The method for treating hyperhidrosis in a patient includes applying the lotion to a scalp of the patient. The lotion may include Schinus terebinthifolius Raddi or Anetholea anista leaf extract or both. The method may include extracting 15 to 20 hair bulbs from the patient, cleaning and treating the hair bulbs for microscopic analysis, using a polarized eye piece for viewing an accumulation of metabolic waste and neutralizing the metabolic waste by applying the lotion to a scalp of the patient.

The present invention comprises various compositions, described in greater detail below, for application to the scalp, in the form of a shampoo, conditioner, and similar products that are applied and subsequently rinsed out (collectively referred to as “shampoos”). Other compositions are applied to the scalp in the form of lotions, salves, or similar materials, which are left on the scalp after application (collectively referred to as “lotions”). These compositions and their methods of use are particularly adapted to treat hair loss caused by excessive sweating, also known as hyperhidrosis.

The compositions include therapeutically effective amounts of various components which work in combination to effectively treat hyperhidrosis to prevent hair loss. While some percentages of the components are described herein, the scope of the invention should not be limited to these preferred amounts, but should include any amounts of the components which are therapeutically effective. The compositions may include active ingredients which promote hair re-growth, pro-biotics, and also hydration components which properly hydrate both scalp and hair. This composition also functions to reduce metabolic waste clogging the hair follicles and disrupting the natural growth cycle. Metabolic waste includes but is not limited to lactic acid, sodium chloride, urea, and potassium ions.

In some embodiments, as described in greater detail below, a user may wash his or her head (scalp and hair) with the shampoo, especially after vigorous physical exertions, to remove waste materials which may inhibit hair growth. In some embodiments, the user may then also apply a lotion to the head to further prevent hair loss, and to also promote hair re-growth.

In one embodiment, the shampoo includes the following ingredients, discussed below. In phase A, about 60.65% water (all percentages are percent weight/total final weight); and about 30.00% surfactant, in this case containing sodium lauryl methyl isethionate, cocamidopropyl betaine, sodium methyl oleoyl taurate, lauryl glucoside, and coco-glucoside. While one embodiment is described herein, these amounts described may vary depending upon the particular needs of a chemist skilled in the art of product formulation for this type of product.

Also included in phase A is amino acid-derived cleansing and skin softening agent such as 1.00-5.00% (preferably about 3.00%) sodium cocoyl glycinate; and 1.00-5.00% (preferably about 3.00%) glycerin-free amphoteric surfactant.

In phase B, 0.05-3.00% (preferably about 0.50%) Vitamin D5 based moisturizer and lubricating compound is included. In one embodiment, this is provided by Panthenol 50% solution F % 12916K, which includes panthenol, capryl hydroxamic acid, 1,2-hexanediol, and propanediol).

Also in phase Bis 0.05-3.00% (preferably about 0.10%) of a saccharomyces ferment.

Also in phase B is 0.01-1.00% (preferably about 0.05% in this embodiment) Caesalpinia spinosa extract (which may include Caesalpinia spinosa fruit pod extract and/or Caesalpinia spinosa fruit extract). The Caesalpinia spinosa extract functions as an antioxidant protective complex for protecting the hair of the user. In this embodiment, the antioxidant protective complex includes Caesalpinia Spinosa Fruit Pod Extract/Caesalpinia spinosa fruit extract, and further includes helianthus Annuus (Sunflower) Sprout Extract, Propylene Glycol, phenoxyethanol, and sodium benzoate.

Also included in phase Bis 0.01-1.00% (preferably about 0.10%) adaptogen which is used for protecting the hair, and which includes propanediol (and) Glycerin (and) Phyllanthus Emblica (Amla) Fruit Extract. Phase B may also include 0.02-5.00% (preferably about 1.00%) of a skin hydration composition, which in this embodiment includes saccharide isomerate, Citric Acid, and Sodium Citrate. In one embodiment, there is about 0.01-1.50% saccharide isomerate, about 0.01-1.50% Citric Acid, and about 0.01-1.50% Sodium Citrate.

Furthermore, phase B also includes 0.05-2.00% (preferably about 0.20%) Pisum sativum (pea) sprout extract. The pea sprout extract may further include about 0.01-1.50% phenoxyethanol and about 0.01-1.50% sodium benzoate.

Also included in phase B is 0.01-1.00% (preferably about 0.10%) of Ocimum basilicum hairy root culture extract. The Ocimum basilicum hairy root culture extract acts as a 5 alpha reductase II inhibitor.

Finally, phase B also includes 0.05-2.00% (preferably about 0.30%) pseudoalteromonas ferment extract, which functions to limit the accumulation of sebaceous lipids and reduce itchiness and flaking. This embodiment may further includes sodium salicylate.

Phase C includes an effective amount of a preservative, in this case about 1.00%, to preserve the composition so that it retains a suitable shelf life.

In the preparation of the final formulation of the shampoo, all phase A ingredients are added to a large mixing vessel and mixing is begun. Phase B ingredients are then added to the mixing vessel, and finally, phase C ingredient is added to the mixing vessel, all while mixing.

As discussed above, the shampoo is applied to wash the head, especially after exercise or physical exertion, to remove sweat and other waste products which may inhibit hair growth. The shampoo is rinsed out so that it does not remain on the head (scalp or hair) in significant amounts following use.

The lotion form of the composition includes the following ingredients: In phase A, 86.93% water (as above, all percentages are percent weight/total final weight); and 0.50% hydroxyethyl cellulose. The hydroxyethyl cellulose is a water soluble polymer with a viscosity range of 3,400-5,000 cps. As noted above, these exemplary ingredients may be adapted to the needs of a chemist skilled in the art of these formulations.

In phase B, 0.01-1.50% (preferably about 0.05%) tetrasodium glutamate diacetate is added as a vegetable-based chelating agent.

In phase C, about 0.01% lactic acid disinfecting and descaling agent.

Phase D includes about 1.00% of a glycerin, sodium hyaluronate, phenoxyethanol, and ethylhexlglycerin composition.

Also in phase D is about 0.01% of hydrolyzed sodium hyaluronate.

Also in phase D is 0.05-5.00% (preferably about 2.00%) of Epilobium angustifolium flower extract. The term “flower extract” is defined, for purposes of this application, to include leaf and stem materials as well as the actual flower. The Epilobium angustifolium flower extract provides antibacterial, anti-inflammatory, and antioxidant properties. While this has been used in various skin care treatments for other uses (e.g., burns and wrinkles), it has not been used in preventing hair loss, and in particular for treating hair loss caused by hyperhidrosis.

Also in phase D is 0.02-5.00 ¾ (preferably about 1.00%) of a saccharomyces ferment.

Also included in phase Dis about 1.00% adaptogen which is used for protecting the hair, and which includes propanediol, glycerin, and Phyllanthus Emblica (Amla) Fruit Extract.

Also included in phase D is 0.02-3.00% (preferably about 0.50%) of Caesalpinia spinosa fruit and pod extract (and) helianthuss annuus (sunflower) sprout extract (and) propylene glycol (and) phenoxyethanol (and) sodium benzoate.

Furthermore, phase D also includes 0.50-5.00% (preferably about 2.00%) Pisum sativum (pea) sprout extract. The pea sprout extract may further include phenoxyethanol and sodium benzoate.

Also included in phase D is about 1.00% of a hair loss reduction formulation that includes basil hairy root culture extract, which acts as a 5 alpha reductase II inhibitor. Other products that utilize basil hairy root culture extract may alternatively be used in other embodiments.

Finally, phase D also includes 0.05-5.00% (preferably about 3.00%) pseudoalteromonas ferment extract, which functions to increase collagen production (collagen I and IV), and also to limit the accumulation of sebaceous lipids and reduce itchiness and flaking. The ferment is typically used along with sodium salicylate, which is an anti-inflammatory agent.

Phase E includes about 1.00% of phenoxyethanol (and) ethylhexlglycerin.

In the preparation of the final formulation, all phase A ingredients are added to a large mixing vessel and mixing is begun. Phase B ingredients are then added to the mixing vessel, and finally, phase D and then phase E ingredient is added to the mixing vessel, all while mixing.

In some embodiments, additional components and compounds may also be added to either the shampoo or the lotion. One such potential addition is anetholea anisata leaf extract, which may be used in any therapeutically effective ratio, typically, 0.05-3.00%, preferably 0.40-2.00%.

Another potential addition is pink peppercorn (Schinus terebinthifolious Raddi), which may be used in any therapeutically effective desired, typically 0.01-1.00%, preferably about 0.20%.

The invention further includes a method for treating hair loss from hyperhidrosis in mammals such as humans. The method includes the steps of providing a shampoo such as described above, and applying the shampoo to the scalp, especially after heavy physical activity, or prior to sleeping. The shampoo is applied to clean, condition, or otherwise treat the hair and scalp of the user, and then rinsed out.

The method includes the additional steps of providing a lotion such as described above, and applying the lotion to the scalp, and left in. The lotion may be applied and then covered with a wrap prior to sleeping, so that the lotion is effective while the user is sleeping.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A method for treating hyperhidrosis in a patient, the method comprising:
preparing a shampoo in a mixing vessel including:
adding a first mixture including:
40-90% water by weight;
10-50% surfactant by weight;
1-5% amino acid derived cleansing and skin softening agent by weight; and
1-5% glycerin-free amphoteric surfactant by weight;
adding and mixing a second mixture including:
0.05-3.00% Vitamin B % moisturizer by weight;
0.05-3.00% saccharomyces ferment by weight;
0.01-1.00% Caesalpinia spinosa extract by weight;
0.01-1.00% of an adaptogen including propanediol, glycerin and Phyllanthus Emblica Fruit extract;
0.05-5.00% of a skin hydration composition by weight;
0.05-5.00% Pisum sativum sprout extract by weight;
0.05-5.00% Ocimum basilicum hairy root culture extract by weight;
and
0.05-5.00% pseudoalteromonas ferment extract by weight; and
adding and mixing 0.1-5.00% of a preservative by weight;
applying the shampoo to a scalp of the patient; and
rinsing the shampoo from the scalp of the patient;
wherein weight percentages are based on total weight of the shampoo.

2. The method of claim 1 wherein the shampoo includes Schinus terebinthifolius Raddi.

3. The method of claim 1 wherein the shampoo includes Anetholea anista leaf extract.

4. The method of claim 3 wherein the shampoo includes Schinus terebinthifolius Raddi.

5. A method for diagnosing and treating hyperhidrosis in a patient using the shampoo of claim 1, the method comprising:
extracting 15 to 20 hair bulbs from the patient;
cleaning and treating the hair bulbs for microscopic analysis;
using a polarized eye piece for viewing an accumulation of metabolic waste; and
neutralizing the metabolic waste with the steps of:
applying the shampoo to the scalp of the patient; and
rinsing the shampoo from the scalp of the patient.

6. The method of claim 5 wherein the shampoo includes Schinus terebinthifolius Raddi.

7. The method of claim 5 wherein the shampoo includes Anetholea anista leaf extract.

8. The method of claim 5 wherein the shampoo includes Schinus terebinthifolius Raddi.

9. A method for treating hyperhidrosis in a patient, the method comprising:
preparing a lotion in a mixing vessel including:
adding a first mixture including:
40-95% water by weight; and
0.05-3.00% hydroxyethylcellulose;
adding and mixing to the mixing vessel 0.01-2.00% tetrasodium glutamate diacetate by weight;
adding and mixing to the mixing vessel 0.01-1.00% lactic acid by weight;
preparing a second mixture including:
a 0.1-5.00% formulation of glycerin, sodium hyaluronate, phenoxyethanol and ethylhexlglycerin by weight;
0.001-1.00% hydrolyzed sodium hyaluronate by weight;
0.05-5.00% Epilobium angustifolium flower extract by weight;
0.02-5.00% saccharomyces ferment by weight;
0.01-1.00% of an adaptogen including propanediol, glycerin and Phyllanthus Emblica Fruit extract by weight;
a 0.02-3.00% formulation of Caesalpinia spinosa extract, helianthuss annuus sprout extract, propylene glycol and sodium benzoate by weight;
0.5-5.00% Pisum sativum sprout extract by weight;
a 0.05-5.00% formulation including Ocimum basilicum hairy root culture extract by weight; and
0.05-5.00% pseudoalteromonas ferment extract by weight; and
adding and mixing the second mixture to the mixing vessel; and
preparing a third mixture including phenoxyethanol and ethylhexlglycerin; and
adding and mixing to the mixing vessel 0.1-5.00% of the third mixture by weight; and
applying the lotion to a scalp of the patient;
wherein weight percentages are based on total weight of the lotion.

10. The method of claim 9 wherein the lotion includes Schinus terebinthifolius Raddi.

11. The method of claim 9 wherein the lotion includes Anetholea anista leaf extract.

12. The method of claim 11 wherein the lotion includes Schinus terebinthifolius Raddi.

13. A method for diagnosing and treating hyperhidrosis in a patient using the lotion of claim 9, the method comprising:
extracting 15 to 20 hair bulbs from the patient;
cleaning and treating the hair bulbs for microscopic analysis;
using a polarized eye piece for viewing an accumulation of metabolic waste; and
neutralizing the metabolic waste with the steps of:
applying the lotion to a scalp of the patient;
wherein weight percentages are based on total weight of the lotion.

14. The method of claim 13 wherein the lotion includes Schinus terebinthifolius Raddi.

15. The method of claim 13 wherein the lotion includes Anetholea anista leaf extract.

16. The method of claim 15 wherein the lotion includes Schinus terebinthifolius Raddi.
